(19) United States
(12) Patent Application Publication
(10) Pub. No.: US 2015/0203893 A1
(43) Pub. Date: Jul. 23, 2015

(54) Concurrently Evaluating Multiple Disease States Via Processing A Bio-Sample With A Single Multi-Channel Micro-Channel Device

(71) Applicant: Analogic Corporation, Peabody, MA (US)

(72) Inventor: John P. O'Connor, Andover, MA (US)

(21) Appl. No.: 14/160,684

(22) Filed: Jan. 22, 2014

(51) Int. Cl. C12Q 1/68

(52) U.S. Cl. CPC C12Q 1/6806

(57) ABSTRACT

A method concurrently evaluates multiple different disease states of a subject with a single multi-channel micro-channel device. The method includes obtaining a bio-sample of the subject. The method further includes concurrently processing a first sub-portion of the bio-sample in a first channel and a second different sub-portion of the bio-sample in a second different channel of the device. The method further includes performing a first comparison of a first result of the processing of the first sub-portion with a first disease profile corresponding to the first disease and a second comparison of a second result of the processing of the second sub-portion with a second disease profile corresponding to the second disease. The method further includes generating a signal indicating a presence or absence of the first disease and a presence or absence of the second disease respectively in response to the first comparison and the second comparison.

### TECHNICAL FIELD

The following generally relates to a micro-channel device and more particularly to concurrently evaluating multiple disease states of a subject through processing a bio-sample of the subject using multiple channels of a single multi-channel micro-channel device.

### BACKGROUND

A micro-channel device includes a micro channel through which a small volume of a fluid is routed for processing. Such devices have been used in biochip, lab-on-a-chip, and other micro-channel based technologies. Micro-channel devices have been used in applications such as DNA sequencing, or determining an order of the nucleotide bases (adenine, guanine, cytosine, and thymine) in a DNA strand. For this, the DNA strands are moved along the channels through multiple, different processing stations. Micro-channel devices include interfaces for receiving reagents, wash solutions, primers, dyes, etc. for the different processing stations and for interfacing with a moving sub-system, which moves the sample through the micro channel from processing station to processing station.

With a DNA sequencer, a sample including DNA is first processed to extract one or more DNA strands from the sample. An extraction fluid such as a lyses reagent is routed to the region via the micro-channels. The DNA strand is then purified. A purification fluid such as a wash solution is routed to the region via the micro-channels. The DNA strand is then replicated and labeled, e.g., via polymerase chain reaction (PCR) or otherwise by a replication sub-system. Replication and labeling fluids such as a primer and fluorescent dyes are routed to the region via the micro-channels. The DNA strand is then separated by nucleotide, e.g., via capillary electrophoresis or otherwise, and analyzed, e.g., via an optical detection system. Sequencing has been determined based on dye wavelength, and a signal indicative of the sequence is read out.

Micro-channel devices have included multiple micro channels. This has allowed sequencing of DNA for multiple different subjects in parallel. One automated DNA sequencer has integrated the above-noted processing sub-systems into a single device. With this sequencer, a micro-channel device is placed in a single position, and each of the sub-systems sequentially processes the biochip at this position within hours or less. Systems have been used to process DNA in an attempt to identify disease states. Unfortunately, these systems are in general specific to only one disease and are not capable of combination with other elements to determine the possible presence of multiple disease states.

### SUMMARY

Aspects of the application address the above matters, and others.

In one aspect, a method concurrently evaluates multiple different disease states of a subject with a single multi-channel micro-channel device. The method includes obtaining a bio-sample of the subject. The bio-sample including a first set of first DNA strands that are indicative of a first disease, and at least a second set of second DNA strands that are indicative of a second different disease. The method further includes processing a first sub-portion of the bio-sample in a first channel of the single multi-channel micro-channel device. The method further includes processing a second different sub-portion of the bio-sample in a second different channel of the single multi-channel micro-channel device, concurrently with the processing of the first sub-portion of the bio-sample in a first channel of the single multi-channel micro-channel device. The method further includes performing a first comparison of a first result of the processing of the first sub-portion with a first disease profile corresponding to the first disease. The method further includes performing a first comparison of at least a second result of the processing of the second sub-portion with a second disease profile corresponding to the second disease. The method further includes generating a signal indicating a presence or absence of the first disease and a presence or absence of the second disease respectively in response to the first comparison and the second comparison.

In another aspect, a micro-channel device includes a first micro channel configured to receive a first sub-portion of a bio-sample of a subject for processing with a first set of disease evaluating processing agents. The micro-channel device further includes a second micro channel configured to receive a second sub-portion of the bio-sample of the subject for processing with a second set of disease evaluating processing agents. The first and the second of disease evaluating processing agents are different. The first set of disease evaluating processing agents corresponds to a first disease. The second set of disease evaluating processing agents corresponds to a second different disease.

In another aspect, a sample processing apparatus is configured to concurrently evaluate multiple different disease states of a subject from a bio-sample supported by a single multi-channel micro-channel device. The single multi-channel micro-channel device includes multiple micro channels. Each micro channel includes its own set of processing agents. The sets of processing agents for at least two of the micro channels are different. The sample processing apparatus comprises: a controller; a plurality of processing stations; a set of disease state evaluation algorithms; and disease profiles. The controller controls the plurality of processing stations to process the bio-samples in the multiple micro channels using respective sets of processing agents based on the set of disease state evaluation algorithms and the disease profiles. The controller also generates a signal indicating whether a disease corresponding to one of the disease state evaluation algorithms is present in the bio-sample.

Those skilled in the art will recognize still other aspects of the present application upon reading and understanding the attached description.

### BRIEF DESCRIPTION OF THE DRAWINGS

The application is illustrated by way of example and not limitation in the figures of the accompanying drawings, in which like references indicate similar elements and in which:

FIG. 1 schematically illustrates an example micro-channel device in connection with a sample processing apparatus;

FIG. 2 schematically illustrates a cross sectional view of the micro-channel device in FIG. 1 which includes a row or layer of lanes of micro channels;

FIG. 3 schematically illustrates components of the micro-channel device illustrated in FIG. 1, including processing station interfaces and agent chambers with processing agents;

FIG. 4 schematically illustrates a cross sectional view of a variation of the micro-channel device in FIG. 1 that includes a two dimensional matrix of lanes of micro channels;

FIG. 5 schematically illustrates a sub-portion of a variation of the micro-channel device of FIG. 1; and

FIG. 6 illustrates a method for concurrently evaluating multiples disease states of a subject with the micro-channel device described herein.

### DETAILED DESCRIPTION

The following describes an approach for concurrently evaluating multiple disease states of a subject through processing a bio-sample of the subject with a single multi-channel micro-channel device. The multiple disease states, in one no-limiting instance, correspond to medical facility or hospital-acquired (or nosocomial) infections. Such infections include an infection whose development is favored by a hospital environment, such as one acquired by a patient during a hospital visit or one developing among hospital staff like fungal and/or bacterial infections. Other disease states are also contemplated herein.

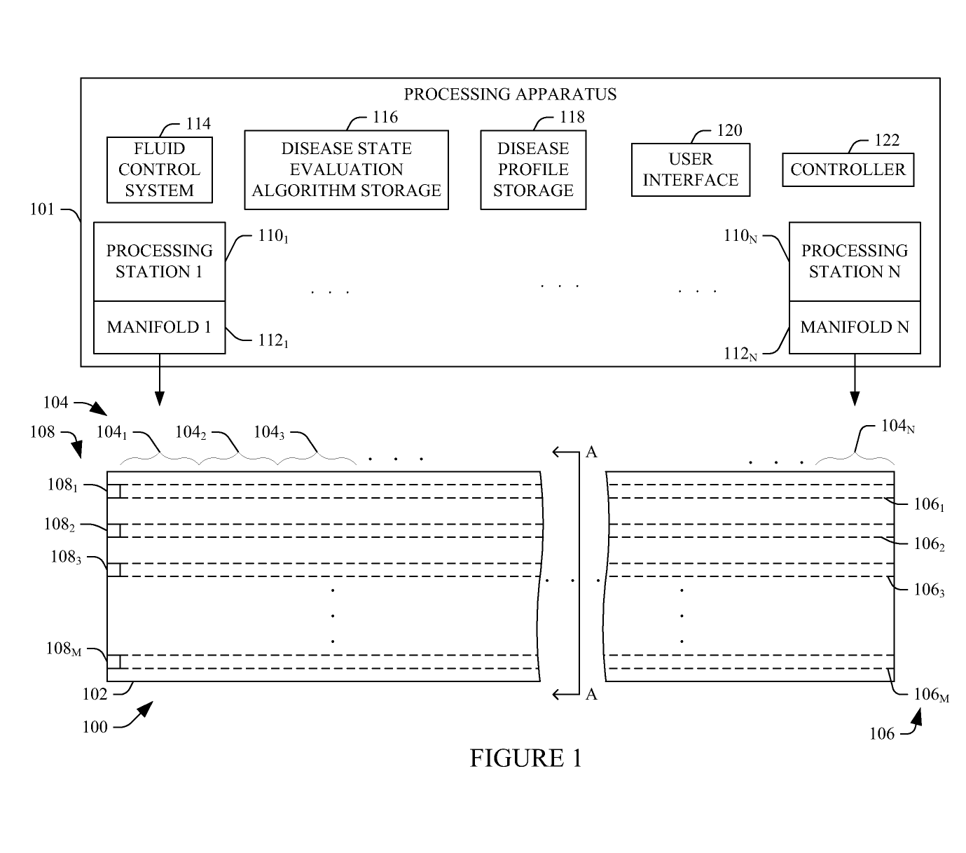

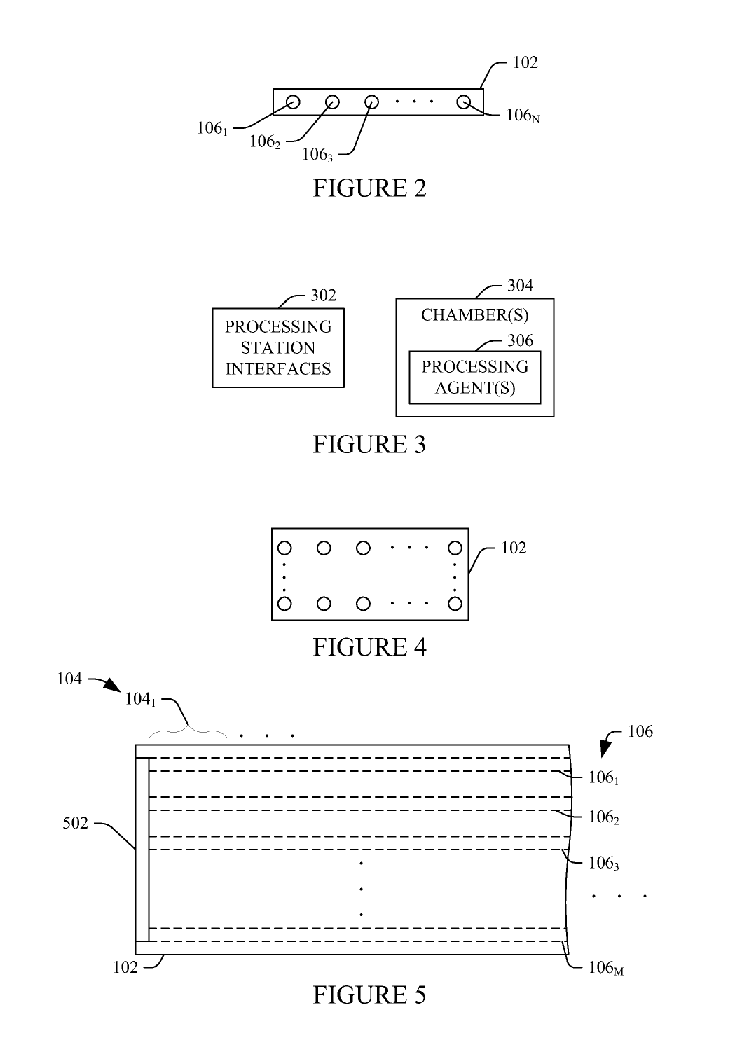

FIGS. 1 and 2 illustrate a micro-channel device 100 in connection with a sample processing apparatus 101. FIG. 1 shows a view looking down on a major surface of the micro-channel device 100, and FIG. 2 shows a cross-sectional view looking into the micro-channel device 100 along line A-A of FIG. 1.

Micro-channel devices, such as the micro-channel device 100, in general, include devices in which a volume of a fluid(s) is transported through one or more micro (e.g., sub-millimeter, nanometer to micron, etc.) channels or capillaries of the device. Examples of such a device include, but are not limited to, a biochip (e.g., for DNA, enzymatic, protein, etc. analysis), a lab-on-a-chip, and/or other micro channel devices.

The micro-channel device 100 includes a substrate 102. The substrate 102 may include glass, silicon, a polymer(s), ceramic, and/or one or more other materials. The substrate 102 includes N processing regions 104₁, 104₂, 104₃, . . . , 104_{N}, where N is an integer equal to or greater than one. The N processing regions are collectively referred to herein as processing regions 104.

Examples of processing at the processing regions 104 include selectively extracting DNA from a sample, replicating (amplifying) the extracted DNA, labelling the replicated DNA, separating the nucleotide bases in the DNA based on the label, sequencing the nucleotide bases, etc. In one instance, the processing is the same across a processing region 104_{α} (where α=1, . . . N). In another instance, the processing may be different across the processing region 104_{α}.

The substrate 102 further includes M micro channels 106₁, 106₂, 106₃, . . . , 106_{M}, where M is an integer equal to or greater than one (e.g., 2, 6, 10, 50, etc.). The M micro channels are collectively referred to herein as micro channels 106. The micro channels 106 route one or more sample fluids (e.g., sputum, blood, etc.) from processing region 104_{α} to a processing region 104_{β} (where β#α, and β=1, . . . N). The micro channels 106 are shown as linear tubes. However, the micro channels 106 can be curved, etc. and/or otherwise shaped (e.g., elliptical, square, etc).

In the illustrated embodiment, each micro channel 106 includes a sample port 108₁, 108₂, 108₃, . . . , 108_{M}, collectively referred to herein as sample ports 108, which are configured to receive samples for processing. For example, each of the sample ports 108 may be configured to receive a sub-portion of a buccal swab that includes a bio-sample or the portion of the bio-sample extracted from the bio-sample. In this configuration, at least two of the sample ports 108 can receive a sub-portion from a same sample or different samples.

Briefly turning to FIG. 3, components of the substrate 102 for an individual processing region 104_{α} of an individual micro channel 106_{δ} (where δ=1, . . . M) are illustrated. As shown, each of the micro channels 106 includes a set of processing station interfaces 302. Each processing station interface 302 includes channels, valves, etc. for communication with a different processing station of the sample processing apparatus 101.

Each of the micro channels 106 also includes one or more chambers 304. The chambers 304 each hold a processing agent 306 such as a reagent, a lysis agent, a detergent, etc., for example, to process DNA samples. The one or more chambers 304 for a processing region 104_{α} can be located within the processing region 104_{α} and/or outside of such region with conduits that route the agent to the processing region 104_{β}. Multiple channels of the micro channels 106 can have the same or different interface 302, chambers 304, and/or agents 306.

Returning to FIGS. 1 and 2, at least two of the channels 106 are configured to process, in parallel or in series, different DNA strands respectively corresponding to individual and different disease states. For example, a first channel 106_{δ} can be used to process a first disease state, a second channel 106_{μ} (where μ≠δ and μ=1, . . . M) can be used to process a second disease state, a third channel 106_{ν} (where ν≠δ or μ and ν=1, . . . M) can be used to process a third disease state, etc. The disease states can be associated with a same individual or different individuals.

Where the same processing is performed at each of the processing regions 104 by each of the processing stations 110 for each of the individual channels 106, the processing components of a processing station 104_{α} are the same for each of the channels 106. Where different processing is performed across at least two of the channels 106 and at least one of the processing regions 104 by at least one of the processing stations 110, the processing components of a processing station 110_{τ} (where τ=1, . . . N) may be different for each of the channels 104.

Additionally or alternatively, the agents and/or parameters of the processing across at least two of the channels 106 at at least one of the processing regions 104 by at least one of the processing stations 110 may be different. For example, different agents may be used for two different channels of the channels 106 at a same processing region 104_{α}, different thermo-cycling patterns and/or temperatures may be used for two different channels of the channels at the same processing region 104_{α}, etc. In the former case, the agents 306 in the chambers 304 will be different for each channel.

In a variation (e.g., FIG. 4), the substrate 102 includes a two-dimensional matrix of the processing regions 104. Other configurations are also contemplated herein.

In another variation (FIG. 5), each of the micro channels 106 interfaces with a shared mixing chamber 502 that receives a same buccal swab or other collection device. In this instance, each of the micro channels 106 has access to the mixing chamber 502 and includes the appropriate agent(s) 306 for extracting a different DNA strand from the collection device.

Returning to FIG. 1, the sample processing apparatus 101 includes N processing stations 110₁, . . . , 110_{N}, collectively referred to herein as processing stations 110. Each of the processing stations 110 can carry out different sub-processing. By way of non-limiting example, stations 110 may be respectively extract a DNA strands from a sample, purify the extracted strands, replicate (amplify) and label the replicated DNA strands, separate the nucleotide bases in the DNA strand(s) based on the label, sequence the nucleotide bases, etc.

The processing stations 110₁, . . . , 110_{N} respectively include manifolds 112₁, . . . , 112_{N}, collectively referred to herein as manifolds 112. The manifolds 112₁, . . . , 112_{N} respectively include interfaces (e.g., channels, valves, etc.) for interfacing the set of processing station interfaces 302 (FIG. 3) of the substrate 102 of the micro-channel device 100. The manifold interfaces 112 for each of the channels 106 at a processing region 104_{α} may be the same or different.

In the illustrated embodiment, the sample processing apparatus 101 further includes a fluid control system 114 that controls (e.g., actuates, etc.) a flow of a sample fluid in a micro channel(s) 106_{δ}. The fluid control system 114 includes a pressure system with a pump, a valve, a sensor, and/or one or more other components. The fluid control system 114 controllably moves the fluid through the micro channel(s) 106_{δ} via pressure from the pressure system.

In a variation, at least a sub-portion of the fluid control system 114 is located on the micro-channel device 100 and includes micro-components such as a micro pump, a micro valve, a micro sensor, and/or one or more other micro components. The micro components can be based on Micro Electro Mechanical Systems (MEMS) or other technology. In an alternative embodiment, the fluids can be passively moved under capillary forces, etc. and/or otherwise.

Disease state evaluation algorithm storage 116 stores a set of algorithms for evaluating different disease states. For example, one algorithm may include instructions for processing the first disease state, another algorithm may include instructions for processing the second disease state, another algorithm may include instructions for processing the third disease state, etc. The algorithms may be user selectable and/or grouped in predefined groups.

Disease profile storage 118 stores profiles for different diseases. The profiles for the different diseases are compared with the processing results to identify whether a disease is present or absent, based on the sample. For example, where a channel 106_{δ} is used to process a first disease based on a disease state evaluation algorithm corresponding to the disease, the results of the processing are compared with a disease profile for the disease to identify whether a disease is present or absent.

A user interface 120 allows a human to interact with the processing apparatus. The user interface 120 may include an input device such as a keyboard, a touchscreen, a mechanical button, and/or other input device(s). The user interface 120 may also include an output device such as a display monitor, audio, etc. In one instance, the user interface 120 allows a user to select a predetermined or other set of algorithms, invoke processing, etc.

A controller 122 controls one or more components of the processing apparatus 101. This may include controlling the processing stations 110 the fluid control system 114, the user interface 120, etc. Such control can be based on user input and/or pre-stored configuration and/or other files.

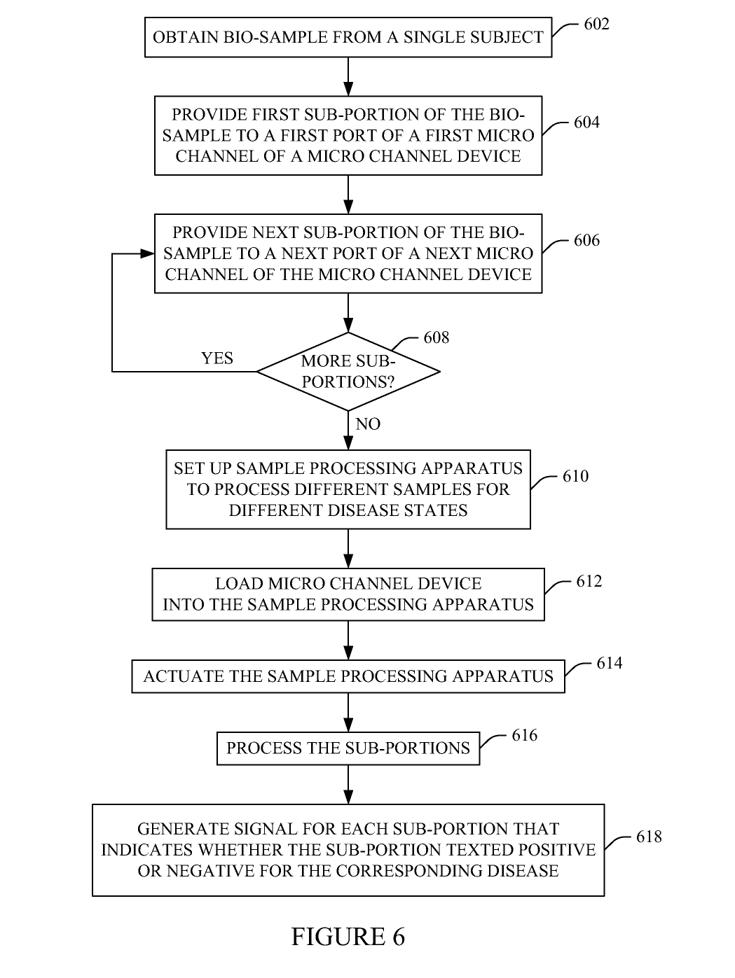

FIG. 6 illustrates a method for concurrently evaluating multiples disease states of a subject with the micro channel device described herein.

At 602, a bio-sample, including DNA, is obtained from a subject. For example, a buccal swab can be used to collect DNA from the cells on the inside of a subject's cheek. Other swabs and non-swabs can also be used to collect DNA from the mouth and/or other regions of the subject.

At 604, a first sub-portion of the bio-sample is inserted or loaded into a first sample port of a first micro channel of a micro-channel device. It is to be understood that “first” in this context does not indicate an ordering such as channel 1 of channels 1-X. Rather, it is the first channel to receive a sample and could be any of one of the X channels.

At 606, a next sub-portion of the bio-sample is inserted or loaded into a next sample port of a next micro channel of the micro-channel device.

At 608, it is determined whether another sub-portion of the bio-sample is to be inserted into a next micro channel of the micro-channel device. If so, then act 606 is repeated for the next sub-portion.

If not, then at 610, a sample processing apparatus is set up to process the loaded sub-portions in at least two different micro channel sample ports using two different processing algorithms, including a first algorithm for evaluating a first disease state and a second algorithm for evaluating a second different disease state.

At 612, the micro channel device is loaded into the sample processing apparatus.

At 614, the sample processing apparatus is actuated to process the sub-portions of the bio-sample.

At 616, the sub-portions of the bio-sample are processed at each sample region of the device via the sample processing stations of the sample processing apparatus. It is to be appreciated that the evaluating can be completed on an order of an hour or more or less.

At 618, the sample processing apparatus generates a signal for each sub-portion that indicates whether the sub-portion texted positive or negative for the corresponding disease.

As discussed herein, in one non-limiting instance, the multiple disease states correspond to medical facility or hospital-acquired (or nosocomial) infections such fungal and bacterial infections. However, in general, any disease which can be evaluated using the systems and/or method described herein are contemplated herein.

The above methods may be implemented by way of computer readable instructions, encoded or embedded on computer readable storage medium, which, when executed by a computer processor(s), cause the processor(s) to carry out the described acts. Additionally or alternatively, at least one of the computer readable instructions is carried by a signal, carrier wave or other transitory medium.

The application has been described with reference to various embodiments. Modifications and alterations will occur to others upon reading the application. It is intended that the invention be construed as including all such modifications and alterations, including insofar as they come within the scope of the appended claims and the equivalents thereof.

What is claimed is:

1. A method for concurrently evaluating multiple different disease states of a subject with a single multi channel micro channel device, comprising:
obtaining a bio-sample of the subject, the bio-sample including a first set of first DNA strands that are indicative of a first disease, and at least a second set of second DNA strands that are indicative of a second different disease;
processing a first sub-portion of the bio-sample in a first channel of the single multi-channel micro-channel device;
processing a second different sub-portion of the bio-sample in a second different channel of the single multi-channel micro-channel device, concurrently with the processing of the first sub-portion of the bio-sample in a first channel of the single multi-channel micro-channel device;
performing a first comparison of a first result of the processing of the first sub-portion with a first disease profile corresponding to the first disease;
performing a first comparison of at least a second result of the processing of the second sub-portion with a second disease profile corresponding to the second disease; and
generating a signal indicating a presence or absence of the first disease and a presence or absence of the second disease respectively in response to the first comparison and the second comparison.

2. The method according to claim 1, wherein at least one of the first or the second disease is indicative of a medical facility acquired infection.

3. The method according to claim 1, wherein the medical facility acquired infection includes a fungal infection.

4. The method according to claim 1, wherein the medical facility acquired infection includes a bacterial infection.

5. The method according to claim 1, wherein the processing of the first and second different sub-portions includes:
selectively extracting the first and second set of first and second DNA strands;
replicating the extracted first and second set of first and second DNA strands;
labelling the replicated first and second set of first and second DNA strands;
separating nucleotide bases in the labelled first and second set of first and second DNA strands;
sequencing the nucleotide bases of the separated first and second set of first and second DNA strands; and
comparing the sequenced nucleotide bases with the first and second disease profiles.

6. The method according to claim 1, wherein the single multi-channel micro-channel device includes at least six different micro channels, and employing each of the at least six different micro channels to evaluate a different disease state of the same subject.

7. The method according to claim 1, wherein the single multi-channel micro-channel device includes at least fifty different micro channels, and employing each of the at least fifty different micro channels to evaluate at least fifty different disease states of the same subject.

8. The method according to claim 1, further comprising:
processing the first sub-portion of the bio-sample in the first channel of the single multi-channel micro-channel device with a first set of reagents, lysis agents, and detergents; and
processing the second sub-portion of the bio-sample in the second channel of the single multi-channel micro-channel device with a second different set of reagents, lysis agents, and detergents,
wherein the first set and the second sets are different sets that includes at least one of a different reagent, lysis agent, or detergent.

9. The method according to claim 8, wherein the first and second sets of reagents, lysis agents, and detergents are located on the micro-channel device.

10. The method according to claim 8, further comprising:
concurrently evaluating multiple the different disease states of the subject with the single multi-channel micro-channel device on an order of an hour.

11. A micro-channel device, comprising:
a first micro channel configured to receive a first sub-portion of a bio-sample of a subject for processing with a first set of disease evaluating processing agents; and
a second micro channel configured to receive a second sub-portion of the bio-sample of the subject for processing with a second set of disease evaluating processing agents;
wherein the first and the second of disease evaluating processing agents are different,
wherein the first set of disease evaluating processing agents corresponds to a first disease, and
wherein the second set of disease evaluating processing agents corresponds to a second different disease.

12. The micro-channel device of claim 11, the first micro channel including: a first set of first chambers respectively storing the first set of disease evaluating processing agents, and the second micro channel including: a second set of second chambers respectively storing the second set of disease evaluating processing agents, wherein the first and the second set of second chambers are different.

13. The micro-channel device of claim 11, each of the micro channels, comprising:
a mechanical interface to a sample processing apparatus, the mechanical interface including one or more of channels or valves.

14. The micro-channel device of claim 11, wherein the first and/or the at least the second disease includes medical facility acquired infections.

15. The micro-channel device of claim 11, wherein the single multi-channel micro-channel device includes at least six different micro channels, each with its own set of chambers respectively storing disease evaluating processing agents for different diseases.

16. The micro-channel device of claim 11, further comprising: a two dimensional matric of micro channels, including the first and the second micro channels.

17. A sample processing apparatus configured to concurrently evaluate multiple different disease states of a subject from a bio-sample supported by a single multi-channel micro-channel device, wherein the single multi-channel micro-channel device with multiple micro channels, each with its own set of processing agents, wherein the sets of processing agents for at least two of the micro channels are different, the sample processing apparatus comprising:
a controller;
a plurality of processing stations;
a set of disease state evaluation algorithms; and
disease profiles,
wherein the controller controls the plurality of processing stations to process the bio-samples in the multiple micro channels using respective sets of processing agents based on the set of disease state evaluation algorithms and the disease profiles, and generates a signal indicating whether a disease corresponding to one of the disease state evaluation algorithms is present in the bio-sample.

18. The sample processing apparatus of claim 17, further comprising:
a plurality of processing stations, each processing station providing a different type of processing.

19. The sample processing apparatus of claim 18, each of the plurality of processing stations, comprising:
a mechanical interface to the single multi-channel micro-channel device, the mechanical interface including one or more of channels or valves.

20. The sample processing apparatus of claim 18, sample processing apparatus, further comprising:
a fluid control system for moving the sample in each channel along the channel from processing station to processing station.
